(19) United States
(12) Patent Application Publication
(10) Pub. No.: US 2014/0066593 A1
(43) Pub. Date: Mar. 6, 2014

(54) PROCESS FOR A FOLATE-TARGETED AGENT

(71) Applicant: ENDOCYTE, INC., (US)

(72) Inventors: Daniel S. RENO, Lafayette, IN (US); Katheryn M. STANFORD, Lafayette, IN (US); Iontcho R. VLAHOV, West Lafayette, IN (US)

(73) Assignee: ENDOCYTE, INC., West Lafayette, IN (US)

(21) Appl. No.: 13/803,150

(22) Filed: Mar. 14, 2013

Related U.S. Application Data
(63) Continuation of application No. 13/698,215, filed on Nov. 15, 2012.
(60) Provisional application No. 61/346,444, filed on May 19, 2010.
(60) Provisional application No. 61/351,022, filed on Jun. 3, 2010.

(51) Int. Cl. C07K 7/02

(52) U.S. Cl. CPC C07K 7/02
USPC 530/329

(57) ABSTRACT

The invention described herein pertains to an improved process for preparing the folate-targeted conjugate EC145 and to the conjugate EC145 prepared using the improved process, as well as to a pharmaceutical composition comprising the conjugate EC145 prepared using the improved process.

### CROSS-REFERENCE TO RELATED APPLICATIONS

This application claims the benefit of U.S. provisional applications 61/346,444, filed 19 May 2010, and 61/351,022, filed 3 Jun. 2010, each of which is incorporated by reference herein.

### TECHNICAL FIELD

The invention described herein pertains to an improved process for preparing the folate-targeted conjugate EC145 and to the conjugate EC145 prepared using the improved process, as well as to a pharmaceutical composition comprising the conjugate EC145 prepared using the improved process.

### BACKGROUND AND SUMMARY OF THE INVENTION

Folate-targeted drugs have been developed and are being tested in clinical trials as cancer therapeutics. EC145 comprises a highly potent vinca alkaloid cytotoxic compound, desacetylvinblastine hydrazide (DAVLBH), conjugated to folate. The EC145 molecule targets the folate receptor found at high levels on the surface of epithelial tumors, including non-small cell lung carcinomas (NSCLC), ovarian, endometrial and renal cancers, and others, including fallopian tube and primary peritoneal carcinomas. It is believed that EC145 binds to tumors that express the folate receptor delivering the vinca moiety directly to cancer cells while avoiding normal tissue. Thus, upon binding, EC145 enters the cancer cell via endocytosis, releases DAVLBH and causes cell death or inhibits cell function. EC145 has the following formula

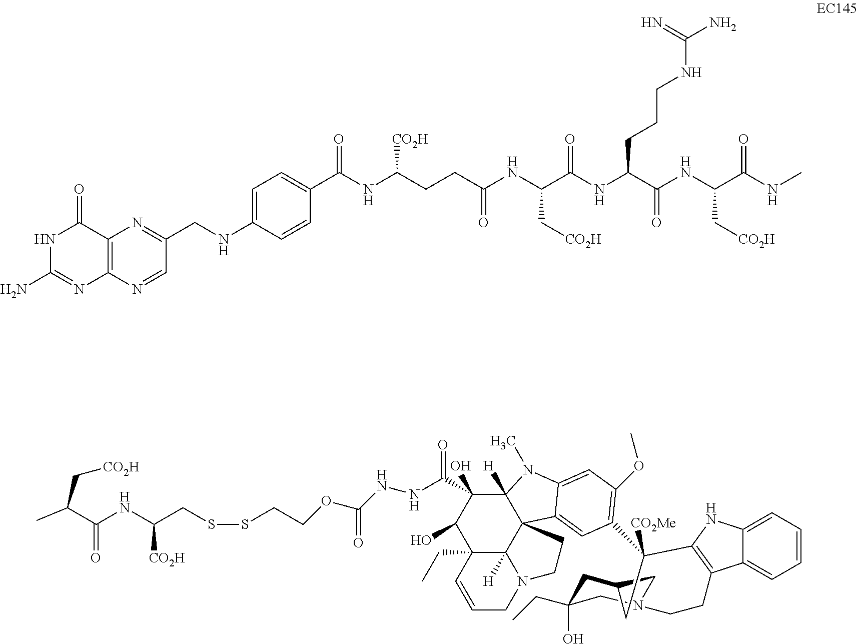

and has been accorded the Chemical Abstracts Registry Number 742092-03-1. As used herein, according to the context, the term EC145 means the compound, or a pharmaceutically acceptable salt thereof; and the compound may be present in a solid, solution or suspension in an ionized form, including a protonated form.

EC145 is disclosed in U.S. Pat. No. 7,601,332, as well as in WO 2007/022493; and particular uses and an aqueous liquid pH 7.4, phosphate-buffered formulation for intravenous administration are disclosed in WO 2011/014821.

The procedures for preparation of EC145 disclosed in U.S. Pat. No. 7,601,332, and in WO 2007/022493 are suitable for preparing EC145 on laboratory scale, e.g. up to tens of milligrams; but problems may arise on increasing the scale.

In the disclosed procedures, EC145 is prepared by forming a disulfide bond using the thiol of formula

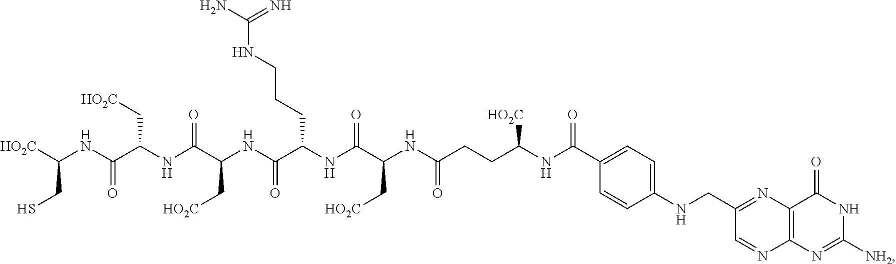 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

known as EC119, with a thiosulfonate- or pyridyldithio-activated vinblastine intermediate. For example, the carbamoyl disulfide intermediate (CDSI) of formula

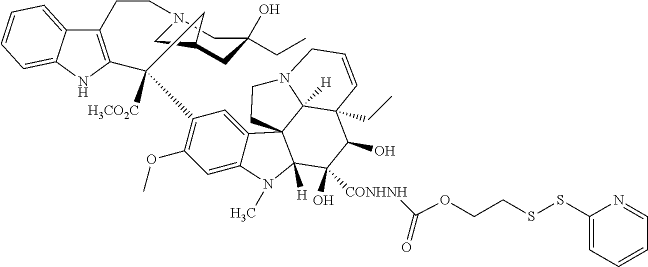

was prepared in dichloromethane (DCM) and isolated following silica gel chromatography. The CDSI was then dissolved in tetrahydrofuran (THF) and added to an aqueous solution of EC119, in which the pH was adjusted with sodium bicarbonate to solubilize the EC119. Upon completion of the reaction, on small scale mixtures (5-10 mL), the THF could be removed using a rotary evaporator; however, foaming under vacuum was so problematic that this operation was not feasible on a larger scale. Accordingly, on completion of the reaction, the mixture was diluted with water, frozen and lyophilized. The crude solid was then dissolved in water and purified by reverse phase chromatography. Chromatographic fractions having greater than 90% peak area of EC145 were combined, diluted with water, and lyophilized. Typically, purified yields were in the 30-40% range with purities in the 90-93% range. The volumes associated with the lyophilizations made preparation of more than gram quantities very cumbersome.

Further, on scale up, it was determined that a portion of the EC145 was being decomposed during the workup of the reaction mixture by the mercaptopyridine liberated in the formation of the disulfide linkage.

It has been determined that this decomposition can be suppressed, and this provides one aspect of the invention. Thus, as one embodiment there is provided a process for preparing EC145 comprising the step of treating a compound of formula

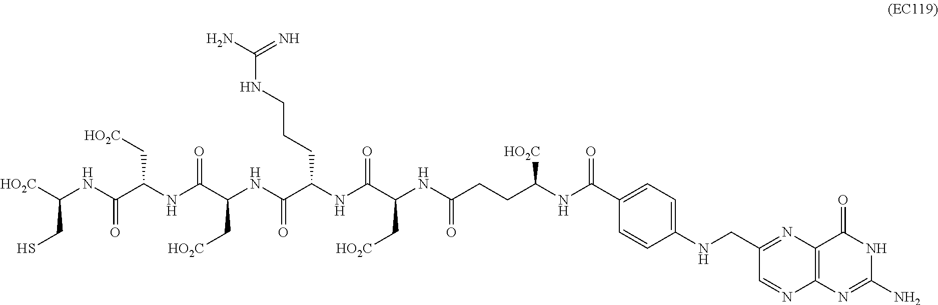 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

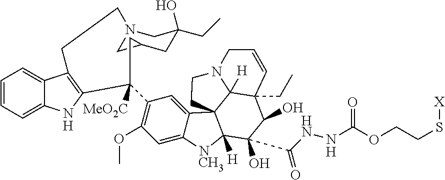

wherein X is alkylsulfonyl, arylsulfonyl, arylthio or heteroarylthio, in the presence of an aqueous buffer of pH less than 8. In one embodiment of the process, X is 2-thiopyridinyl or 3-nitro-2-thiopyridinyl. In one embodiment of the process, X is 2-thiopyridinyl.

For any of the above, in one embodiment, the buffer has a pH of less than about 7. In another embodiment, the buffer has a pH of less than 6.5. In another embodiment, the buffer has a pH of 5.9 to 6.3. In another embodiment, the buffer has a pH of 5.9 to 6.1.

For any of the above, in one embodiment, the buffer is a phosphate buffer. In another embodiment, the buffer is a sodium phosphate buffer. The use of a buffer, as disclosed herein affords better control of the pH and the problem of degradation than the use of sodium bicarbonate.

Another embodiment of the above process comprises the step of treating a compound of formula

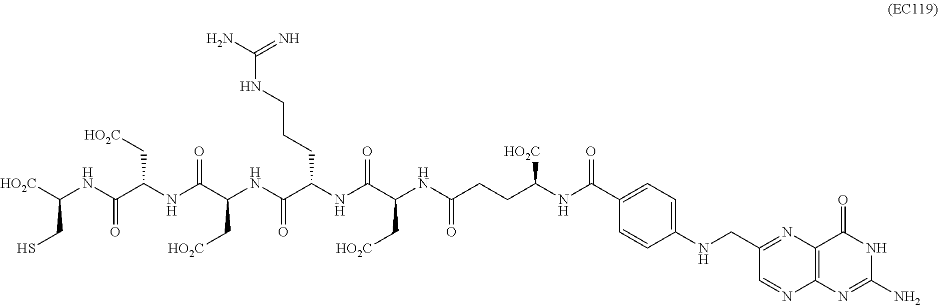 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

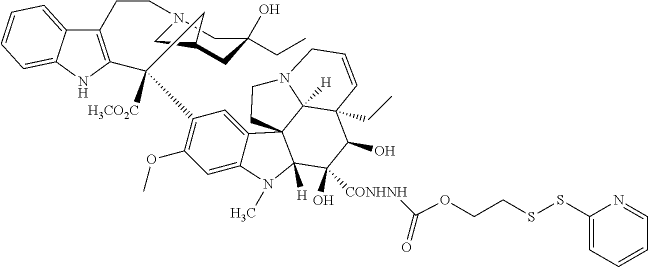

in the presence of a sodium phosphate buffer having a pH of 5.9 to 6.3.

For any of the above, one embodiment is the process wherein the treatment occurs in a liquid medium comprising acetonitrile.

For any of the above, a further aspect is a process further comprising the step of treating desacetylvinblastine hydrazide with an acylating agent of formula Y—CO—O—(CH₂)₂—S—X, or an acid addition salt thereof, wherein Y is a leaving group, to form a reaction mixture comprising the compound of formula

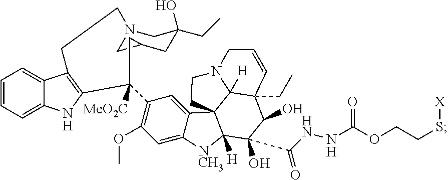

and directly treating the compound of formula

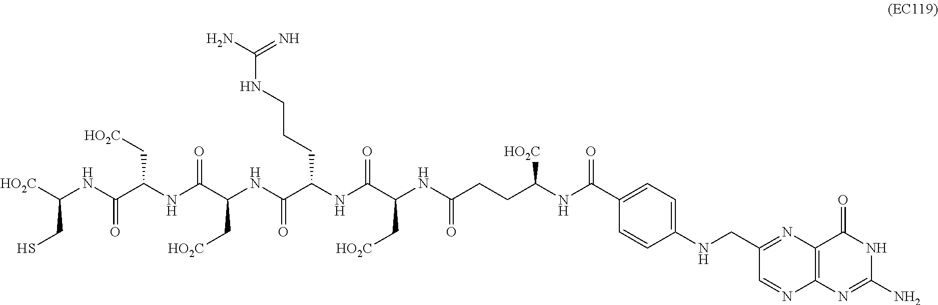 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with the reaction mixture without isolating the compound of formula

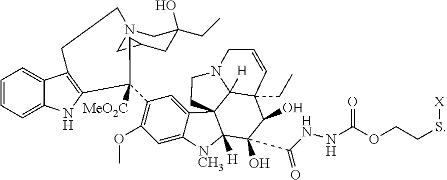

The leaving group Y may be any of a number of leaving groups appropriate for the acylation of the hydrazide. In one embodiment, Y is the residue of an alcohol which forms an active ester with a carboxylic acid or with the monoester of a carbonic acid, for example a 4-nitrophenoxy residue, a 1-benzotriazolyloxy residue or a 7-azabenzotriazol-1-yloxy residue. In one embodiment, the acylating agent is of the formula

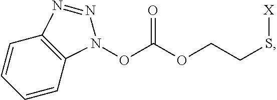

or an acid addition salt thereof. In another embodiment, the acylating agent is of the formula

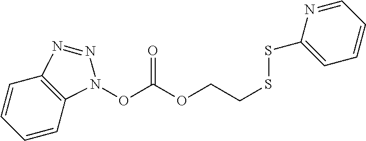 [SMILES: O=C(OCCSSc1ccccn1)On1nnc2ccccc21]

and is introduced in the form of an acid addition salt. When the acylating agent is introduced in the form of an acid addition salt, a base such as triethylamine or diisopropylethylamine is used to free the base. In another embodiment, the acylating agent is of the formula

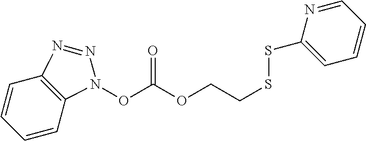 [SMILES: O=C(OCCSSc1ccccn1)On1nnc2ccccc21]

and is introduced in the form of the free base.

For any of the above, one embodiment is the process wherein the desacetylvinblastine hydrazide is treated with the acylating agent in a solvent comprising acetonitrile.

For any of the above, one embodiment is the process wherein the desacetylvinblastine hydrazide is provided in a highly purified form. As described in the examples below, desacetylvinblastine hydrazide may be obtained as a highly purified solid using a procedure involving dissolution/precipitation (denoted as crystallization) from ethyl acetate and toluene.

For any of the above one embodiment is the process wherein the EC119 is provided in a highly purified form. EC119 is synthesized using Fmoc-based solid phase chemistry. First Fmoc-Cys(Trt)-OH is loaded onto the resin through esterification with 2-chlorotrityl chloride resin in the presence of diisopropylethylamine (DIPEA). The Fmoc protecting group on the resin-bound Cys(Trt) is then removed by treating the resin with 6% piperazine in 0.1 M HOBt in dimethylformamide (DMF). The resin is washed with DMF and methyl t-butyl ether (MTBE). Fmoc-Asp(OtBu)-OH is coupled to the resin with N,N′-diisopropylcarbodiimide (DIC) and N-hydroxybenzotriazole (HOBt). The coupling reaction is monitored by a Kaiser test. The deprotection and coupling are repeated with Fmoc-Asp(OtBu)-OH, Fmoc-Arg(Pbf)-OH, Fmoc-Asp(OtBu)-OH and Fmoc-Glu-OtBu. The coupling of N¹⁰-TFA-Pte-OH uses 1.2 equivalents of N¹⁰-TFA-Pte, 1.2 equivalents of PyBOP, 1.2 equivalents of HOBt and 2.4 equivalents of DIPEA. The trifluoroacetyl group is removed with 2% hydrazine in DMF. The peptide is cleaved from the resin with a cleaving reagent containing approximately 85% trifluoroacetic acid, 10% ethanedithiol, 2.5% triisopropylsilane, and 2.5% deionized water. This reaction also results in simultaneous removal of the t-Bu, Pbf, and trityl protecting groups. Crude product is precipitated with MTBE and isolated by filtration. The purity of crude EC119 is approximately 90%. The preparation is described in more detail in the examples.

For any of the above, one embodiment is the process wherein the step of treating desacetylvinblastine hydrazide with an acylating agent to form a reaction mixture comprising the compound of formula

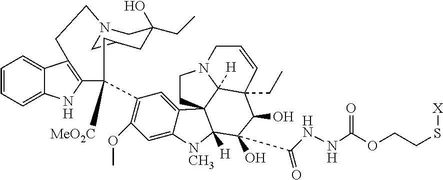

and the step of treating EC119 with the reaction mixture are carried out in the same reaction vessel.

For any of the above, one embodiment is the process further comprising the step wherein the reaction mixture containing EC145 is diluted with citrate buffered, aqueous sodium chloride solution and loaded onto a polystyrene-divinylbenzene polymeric resin column or cartridge for purification. This process makes the dilute and load sequence possible. The dilute and load approach involves diluting the reaction mixture with buffered saline (targeting a 10% acetonitrile content for the diluted solution) and loading this solution onto the chromatography column. This eliminates the need for one ultra-filtration sequence and saves about 12 to 24 hours in processing time. Another embodiment further comprises eluting the EC145 product from the column or cartridge using a mobile phase comprising acetonitrile and citrate buffered, aqueous sodium chloride solution. The use of buffered saline mobile phases improves the chromatographic process in several ways. First, the increased ionic strength of the buffered saline mobile phase influences the partitioning of the product between the mobile and stationary phases. The affinity of the product for the stationary phase is increased to the point that the column's capacity for crude EC145 is more than doubled. The increased affinity of the stationary phase also eliminates the occurrence of product break-through (a portion of the product passing through the column during the loading operation) while loading the crude EC145 onto the column. The higher ionic strength also improves the kinetics of the chromatographic process, affording a chromatographic peak shape that is more Gaussian like and making identification of fraction cut points easier. The inclusion of sodium chloride in the mobile phase also results in reproducible retention volumes and product bandwidths.

For any of the above, one embodiment is the process further comprising the step of using ultra-filtration to afford EC145 as a purified product in aqueous solution. This process avoids the time and product purity loss (about 1%) associated with large scale lyophilization. In addition, it provides the purified product in aqueous solution in a condition appropriate for the further embodiment of filtering through a 0.2 micron absolute filter, which reduces the microbial count and endotoxin levels relative to the process without the filtration.

It has been determined that a potential problem in the process as described in any of the embodiments herein is degradation of EC145 by oxygen. For any of the above, one embodiment is the process wherein the water used in any step contains dissolved oxygen at a concentration that does not exceed about 0.9 parts per million (ppm).

As one aspect of the invention, there is provided as one embodiment the conjugate EC145 prepared by a process described hereinabove. One embodiment is the conjugate EC145 prepared by a process comprising the step of treating a compound of formula

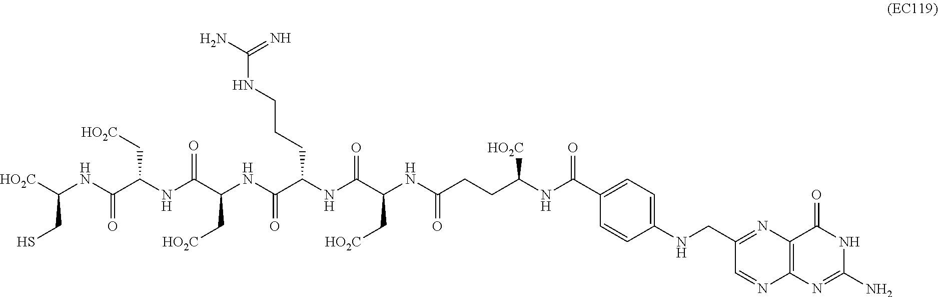 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

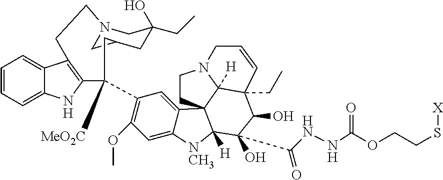

wherein X is alkylsulfonyl, arylsulfonyl, arylthio or heteroarylthio, in the presence of an aqueous buffer wherein the buffer has a pH of 5.9 to 6.3. In one embodiment of the above, X is 2-thiopyridinyl. In a further embodiment of the above conjugate, the process further comprises the step of treating desacetylvinblastine hydrazide with an acylating agent of formula Y—CO—O—(CH₂)₂—S—X, or an acid addition salt thereof, wherein Y is a leaving group, to form a reaction mixture comprising the compound of formula

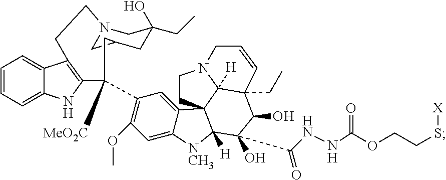

and directly treating the compound of formula

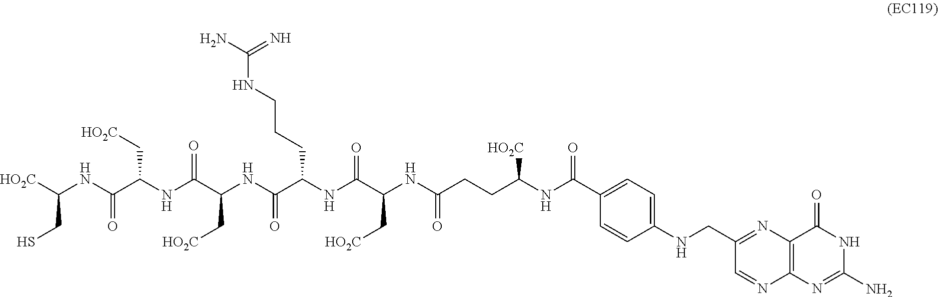 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with the reaction mixture without isolating the compound of formula

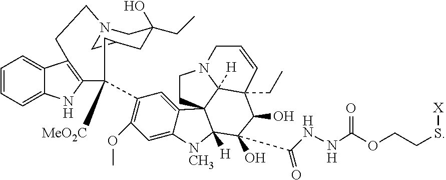

A further embodiment of the above is the conjugate 24 wherein the acylating agent is of the formula

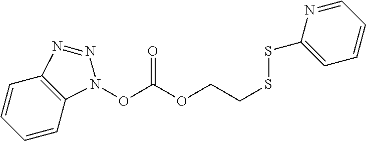 [SMILES: O=C(OCCSSc1ccccn1)On1nnc2ccccc21]

and is introduced in the form of the free base.

As one aspect of the invention, there is provided as one embodiment a pharmaceutical composition comprising the conjugate EC145 as described in any of the above embodiments together with a diluent, excipient or carrier.

### DETAILED DESCRIPTION

Embodiments of the invention are further described by the following enumerated clauses:

1. A process for preparing EC145 comprising the step of treating a compound of formula

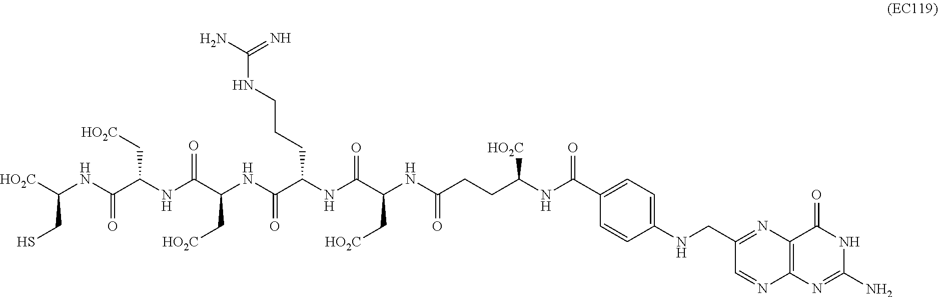 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

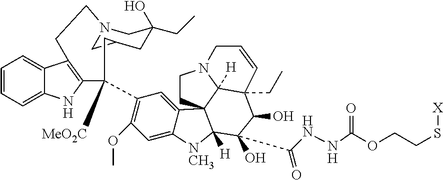

wherein X is alkylsulfonyl, arylsulfonyl, arylthio or heteroarylthio, in the presence of an aqueous buffer of pH less than 8.

2. The process of clause 1 wherein X is 2-thiopyridinyl or 3-nitro-2-thiopyridinyl.

3. The process of clause 1 wherein X is 2-thiopyridinyl.

3.1 The process of any of clauses 1-3 wherein the buffer has a pH of less than about 7.

3.2 The process of any of clauses 1-3 wherein the buffer has a pH of less than 6.5.

4. The process of any of clauses 1-3 wherein the buffer has a pH of 5.9 to 6.3.

5. The process of clause 4 wherein the buffer has a pH of 5.9 to 6.1.

6. The process of any of clauses 1-5 wherein the buffer is a phosphate buffer.

7. The process of clause 6 wherein the buffer is a sodium phosphate buffer.

8. The process of clause 1 comprising the step of treating a compound of formula

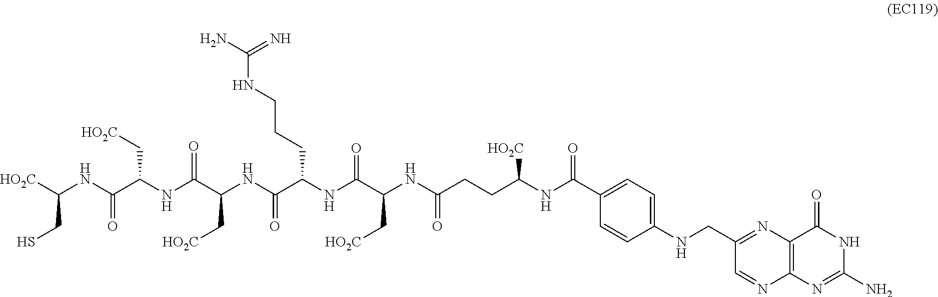 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

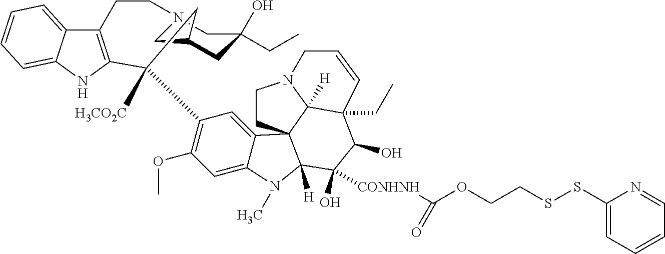

in the presence of a sodium phosphate buffer having a pH of 5.9 to 6.3.

9. The process of any of clauses 1-8 wherein the treatment occurs in a liquid medium comprising acetonitrile.

10. The process of any of clauses 1-9 further comprising the step of treating desacetylvinblastine hydrazide with an acylating agent of formula Y—CO—O—(CH₂)₂—S—X, or an acid addition salt thereof, wherein Y is a leaving group, to form a reaction mixture comprising the compound of formula

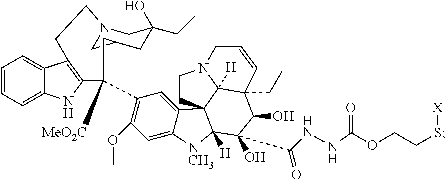

and directly treating the compound of formula

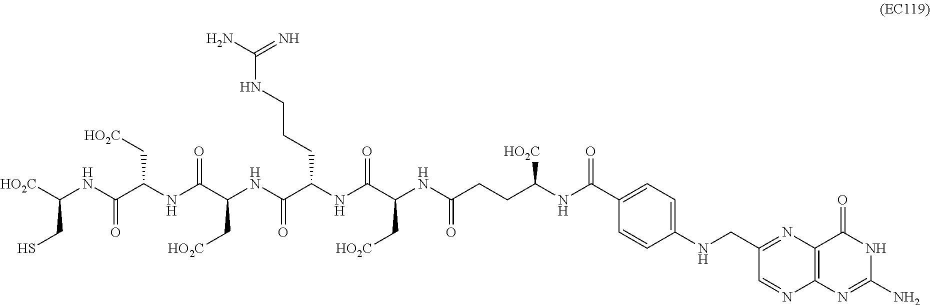 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with the reaction mixture without isolating the compound of formula

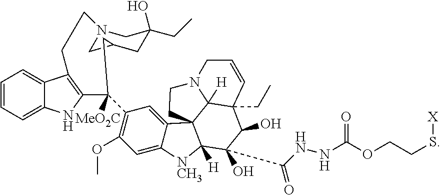

10.1 The process of clause 10 wherein Y is the residue of an alcohol which forms an active ester with a carboxylic acid or with the monoester of a carbonic acid.

10.2 The process of clause 10 wherein Y is a 4-nitrophenoxy residue, a 1-benzotriazolyloxy residue or a 7-azabenzotriazol-1-yloxy residue.

11. The process of clause 10 wherein the acylating agent is of the formula

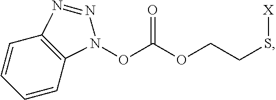

or an acid addition salt thereof.

12. The process of clause 11 wherein the acylating agent is of the formula

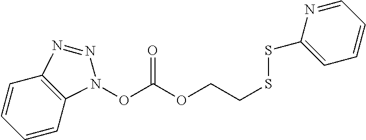 [SMILES: O=C(OCCSSc1ccccn1)On1nnc2ccccc21]

and is introduced in the form of an acid addition salt.

13. The process of clause 11 wherein the acylating agent is of the formula

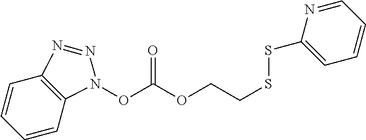 [SMILES: O=C(OCCSSc1ccccn1)On1nnc2ccccc21]

and is introduced in the form of the free base.

14. The process of any of clauses 10-13 wherein the desacetylvinblastine hydrazide is treated with the acylating agent in a solvent comprising acetonitrile.

15. The process of any of clauses 10-14 wherein the desacetylvinblastine hydrazide is provided in a highly purified form.

16. The process of any of clauses 10-15 wherein the step of treating desacetylvinblastine hydrazide with an acylating agent to form a reaction mixture comprising the compound of formula

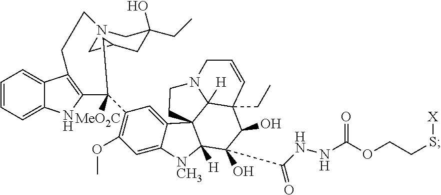

and the step of treating EC119 with the reaction mixture are carried out in the same reaction vessel.

17. The process of any of clauses 1-16, further comprising the step wherein the reaction mixture containing EC145 is diluted with citrate buffered, aqueous sodium chloride solution and loaded onto a polystyrene-divinylbenzene polymeric resin column or cartridge for purification.

18. The process of clause 17 further comprising eluting the EC145 product from the column or cartridge using a mobile phase comprising acetonitrile and citrate buffered, aqueous sodium chloride solution.

19. The process of any one of clauses 1-18 further comprising the step of using ultra-filtration to afford EC145 as a purified product in aqueous solution.

20. The process of any one of clauses 1-19 wherein the water used in any step contains dissolved oxygen at a concentration that does not exceed about 0.9 parts per million (ppm).

21. The conjugate EC145 prepared by a process described in any of clauses 1-20.

22. The conjugate EC145 prepared by a process comprising the step of treating a compound of formula

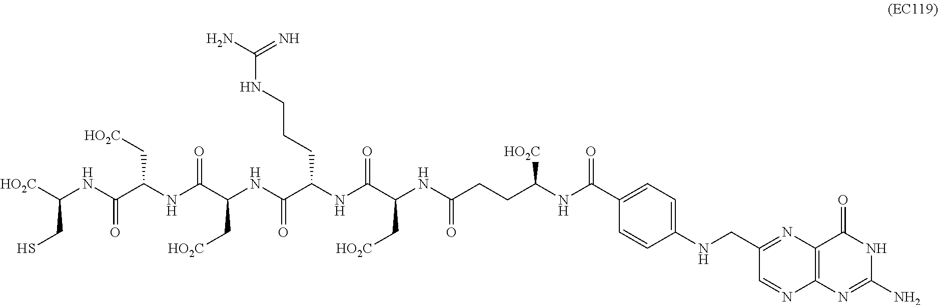 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with a compound of formula,

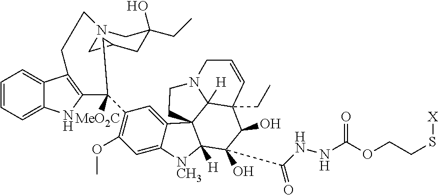

wherein X is alkylsulfonyl, arylsulfonyl, arylthio or heteroarylthio, in the presence of an aqueous buffer wherein the buffer has a pH of 5.9 to 6.3.

23. The conjugate of clause 22 wherein X is 2-thiopyridinyl.

24. The conjugate of clause 22 or 23 wherein the process further comprises the step of treating desacetylvinblastine hydrazide with an acylating agent of formula Y—CO—O—(CH₂)₂—S—X, or an acid addition salt thereof, wherein Y is a leaving group, to form a reaction mixture comprising the compound of formula

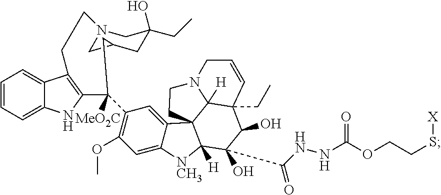

and directly treating the compound of formula

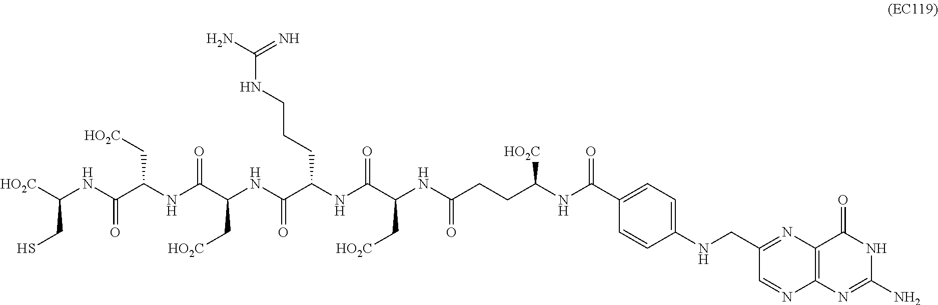 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

with the reaction mixture without isolating the compound of formula

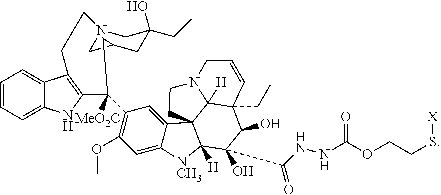

25. The conjugate of clause 24 wherein the acylating agent is of the formula

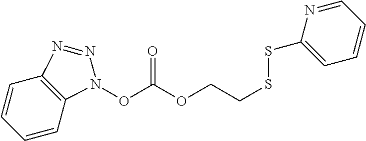 [SMILES: O=C(OCCSSc1ccccn1)On1nnc2ccccc21]

and is introduced in the form of the free base.

26. A pharmaceutical composition comprising the conjugate EC145 as described in any of clauses 21-25 together with a diluent, excipient or carrier.

As used herein, the term “alkyl” includes a chain of carbon atoms, which is optionally branched or cyclic, and which is optionally substituted or may contain an oxygen, sulfur or nitrogen atom, as a heteroalkyl. It is to be further understood that in certain embodiments, alkyl is advantageously of limited length, including C₁-C₆, and C₁-C₄. Illustrative alkyl groups are, but not limited to, methyl, ethyl, n-propyl, isopropyl, n-butyl, isobutyl, tert-butyl, cyclohexyl and the like.

As used herein, the term “aryl” includes monocyclic and polycyclic aromatic carbocyclic groups, each of which may be optionally substituted. Illustrative aromatic carbocyclic groups described herein include, but are not limited to, phenyl, naphthyl, and the like. As used herein, the term “heteroaryl” includes aromatic heterocyclic groups, each of which may be optionally substituted. Illustrative aromatic heterocyclic groups include, but are not limited to, 2-pyridinyl, 3-nitro-2-pyridinyl, and the like.

The term “optionally substituted” as used herein includes the replacement of hydrogen atoms with other functional groups on the radical that is optionally substituted. Such other functional groups illustratively include, but are not limited to, halo, nitro, and the like.

A pharmaceutical composition as described herein means a pharmaceutical composition adapted for the parenteral administration of EC145.

### EXAMPLES

The following examples further illustrate specific embodiments of the invention; however, the following illustrative examples should not be interpreted in any way to limit the invention. Commonly used abbreviations for e.g., solvents, reagents and protecting groups, are used herein. CDSI is used to denote the carbamoyl disulfide intermediate (4).

HPLC Methods used for fraction and sample evaluation in the examples include the following:

### EC145-CMC-IP-0001

Sample preparation: dilute material to approximately 0.5 mg/mL with 8 M guanidine HCl.

### Column: Waters XBridge BEH C18, 3.5 μm, 2.1×100 mm.

Mobile Phases: A) 500 mM ammonium bicarbonate, pH 9.2; B) 75:25 acetonitrile-methanol.
Injection volume: 10 μL
UV detection: 280 nm
Column temperature: 50° C.
Sample temperature 5° C.

| | Time (min) | Flow (mL/min) | % A | % B |
|---|---|---|---|---|
| Gradient: | 0.0 | 0.55 | 95 | 5 |
| | 0.5 | 0.55 | 95 | 5 |
| | 1.0 | 0.55 | 80 | 20 |
| | 5.0 | 0.55 | 73.5 | 26.5 |
| | 21.0 | 0.55 | 71.5 | 28.5 |
| | 27.0 | 0.55 | 70 | 30 |
| | 29.0 | 0.55 | 55 | 45 |
| | 30.0 | 0.55 | 30 | 70 |
| | 33.0 | 0.55 | 30 | 70 |
| | 33.1 | 0.75 | 95 | 5 |
| | 40.0 | 0.75 | 95 | 5 |

EC145-CMC-AM-0001 (version 2.3)
Sample preparation: dilute material to approximately 1 mg/mL with phosphate buffered saline or 1:1 acetonitrile-water (v/v).

### Column: Waters Symmetry C18, 3.5 μm, 4.6×75 mm.

Mobile Phases: A) 10 mM triethylammonium acetate, pH 7.5; B) acetonitrile.
Injection volume: 10 μL
UV detection: 280 nm
Column temperature: 25° C.
Sample temperature 5° C.

| | Time (min) | Flow (mL/min) | % A | % B |
|---|---|---|---|---|
| Gradient: | 0.0 | 1.0 | 85 | 15 |
| | 20.0 | 1.0 | 50 | 50 |
| | 25.0 | 1.0 | 20 | 80 |
| | 30.0 | 1.0 | 20 | 80 |
| | 31.0 | 1.0 | 85 | 15 |
| | 41.0 | 1.0 | 85 | 15 |

### Example 1

### Preparation of EC119

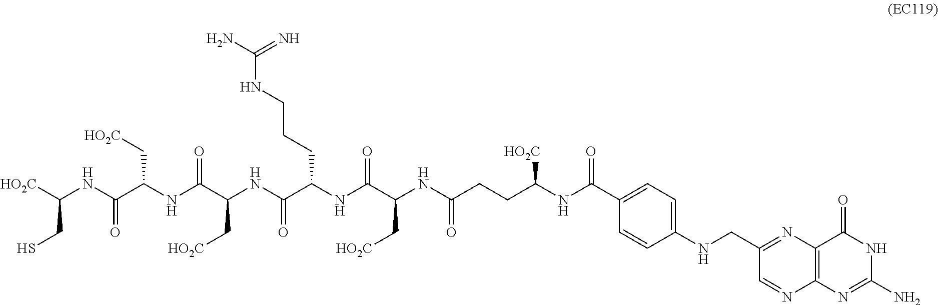 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]

EC119 is synthesized using Fmoc-based solid phase chemistry as follows:

### 1^{st} Coupling

Add 2-chlorotrityl chloride resin to a peptide synthesis vessel. Swell in DMF (10 mL/g resin). Wash with DMF 2 times (10 mL/g resin). Add 0.8 equivalent of Fmoc-Cys(Trt)-OH in DCM/DMF. Add 2 equivalents of DIPEA. Stir for 30 min. Add methanol (1 mL/g resin) and stir for 10 min. Wash with DMF 3 times. Wash with MTBE 3 times. Wash with DMF 3 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test to confirm completion of the coupling.

### 2^{nd} Coupling

Wash with DMF 3 times (10 mL/g resin). Add 2 equivalents of Fmoc-Asp(OtBu)-OH in DMF. Add 2 equivalents of HOBt in DMF. Add 2 equivalents of DIC. Stir for 1.5-3 h. Confirm the coupling with Kaiser test. Wash with MTBE 2 times. Wash with DMF 2 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test.

### 3^{rd} Coupling

Wash with DMF 3 times. Add 2 equivalents of Fmoc-Asp(OtBu)-OH in DMF. Add 2 equivalents of HOBt in DMF. Add 2 equivalents of DIC. Stir for 1.5-3 h. Confirm the coupling with Kaiser test. Wash with MTBE 2 times. Wash with DMF 2 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test.

### 4^{th} Coupling

Wash with DMF. Add 2 equivalents of Fmoc-Arg(Pbf)-OH in DMF. Add 2 equivalents of HOBt in DMF. Add 2 equivalents of DIC. Stir for 1.5-3 h. Confirm the coupling with Kaiser test. Wash with MTBE 2 times. Wash with DMF 2 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test.

### 5^{th} Coupling

Wash with DMF 3 times. Add 2 equivalents of Fmoc-Asp(OtBu)-OH in DMF. Add 2 equivalents of HOBt in DMF. Add 2 equivalents of DIC. Stir for 1.5-3 h. Confirm the coupling with Kaiser test. Wash with MTBE 2 times. Wash with DMF 2 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test.

### 6^{th} Coupling

Wash with DMF 3 times. Add 2 equivalents of Fmoc-Glu-OtBu in DMF. Add 2 equivalents of HOBt in DMF. Add 2 equivalents of DIC. Confirm the coupling with Kaiser test. Wash with MTBE 2 times. Wash with DMF 2 times. Add 6% piperazine in 0.1M HOBt in DMF and stir for 10-20 min. Add 6% piperazine in 0.1M HOBt in DMF and stir for 10-20 min. Wash with DMF 3 times. Wash with MTBE 3 times. Perform Kaiser test.

### 7^{th} Coupling

Wash with DMF 3 times. Add 1.2 equivalents of N¹⁰-TFA-Pte-OH in minimum amount of DMSO. Add 1.2 equivalents of HOBt in DMF. Add 1.2 equivalents of PyBOP in DMF. Add 2.4 equivalents of DIPEA. Stir for 3-5 h. Confirm the coupling with Kaiser test. Wash with DMF 2 times. Wash with MTBE 2 times.

Deprotecting—Removal of Trifluoroacetyl Group

Wash with DMF 2 times. Add 2% hydrazine in DMF and stir for 5 min. Add 2% hydrazine in DMF and stir for 5 min. Add 2% hydrazine in DMF and stir for 5 min. Wash with DMF 3 times. Wash with MTBE 3 times. Dry the resin under vacuum at room temperature.

Cleaving from the Resin

Add cleaving reagent (10 mL/g resin) containing 85% TFA, 2.5% triisopropylsilane, 2.5% water and 10% ethanedithiol to a flask. Cool the mixture in an ice-bath. Add the resin and allow to react for 2-3 hours at room temperature. Filter and collect the filtrate. Add the filtrate to cold MTBE (10 mL of MTBE per 1 mL of filtrate). Stir at 0-5° C. for 30±10 min. Filter the precipitated product through a medium porosity glass filter. Wash the precipitate with cold MTBE 3 times. Dry the product under vacuum at room temperature. Store under nitrogen at −20° C.

### Purification

Crude EC119 is purified by preparative HPLC using a reverse phase C18 column (6-inch column, 2.8 kg, 10 μm, 100 Å). The mobile phases are 0.5% NH₄OAc (A) and 0.5% NH₄OAc/ACN (1:4) (B). 40 g of the crude EC119 is dissolved in 1-5% TFA, filtered through a 1 μm glass fiber filter and load on the 6-inch column. Fractions are collected and sampled for HPLC analysis. The pH of each fraction is adjusted to 3-4 immediately after collection using 50% AcOH under nitrogen to precipitate the product. The precipitated product is centrifuged, washed with 0.1% AcOH and stored at 2-8° C. until further processing. The containers are blanked with nitrogen during centrifugation operation to reduce the potential for oxidation. The pool criteria are purity 98%, isomers of D-Arg⁴, D-Glu² and D-Asp³≦0.25%, other impurity 0.5%. The isomers of D-Asp⁵, D-Asp⁶ and D-Cys cannot be removed by Prep-HPLC and should be suppressed in the synthesis process. The materials that meet the pool criteria are lyophilized as soon as possible (the EC119 solution and the wet precipitate are not stable). The purity of the final product is greater than 98%. The overall yield of pure EC119 including solid phase synthesis and purification is approximately 40%. The product is packed in an amber glass bottle under nitrogen and stored at −20° C.

### Example 2

### A. Typical Conversion of Vinblastine Sulfate into Desacetylvinblastine Hydrazide

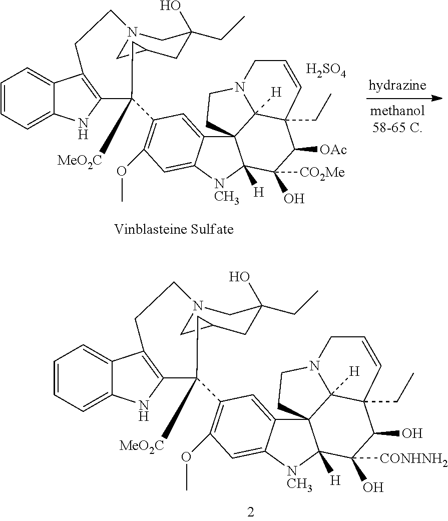

### Materials

Vinblastine Sulfate: USP; FW=909.05 g/mole; Methanol: anhydrous; Hydrazine: anhydrous; FW=32 g/mol; De-ionized water; Ethyl acetate: LC/GC grade; Toluene: LC/GC grade; Monobasic sodium phosphate: ≧99.0%; FW=120 g/mole; Dibasic sodium phosphate: ≧99.0%; FW=142 g/mole; Sodium chloride: reagent grade; FW=58.4 g/mole; Sodium sulfate: anhydrous; 5-norbornen-2-carboxylic acid.

### Procedure

The reaction, extractive work-up and isolation are run under a nitrogen or argon atmosphere. Pressure filters are used to remove the sodium sulfate and capture the product. The sodium chloride solutions used in the quench and wash are sparged with nitrogen or argon until the dissolved oxygen level is not more than 0.9 ppm.

Vinblastine sulfate and anhydrous methanol are charged to an argon purged reactor. 5-Norbornene-2-carboxylic acid and anhydrous hydrazine are added to the reactor. The mixture is stirred, and after the solids dissolve, heat the mixture to around 60° C. By HPLC analysis, when the reaction is complete, it is cooled, quenched and extracted into ethyl acetate. After drying, the product is crystallized from ethyl acetate and toluene. The solids are dried under vacuum overnight at room temperature.

The buffered NaCl contains: 10.0 g NaCl, 7.10-7.30 g NaH₂PO₄, 4.40-4.60 g of Na₂HPO₄ and 90 mL of water. The solution is sparged with argon or nitrogen (dissolved oxygen content <0.9 ppm).

A typical isolated yield is 50-60% of the theoretical maximum.

### B. Steps 2 and 3 of the EC145 Process

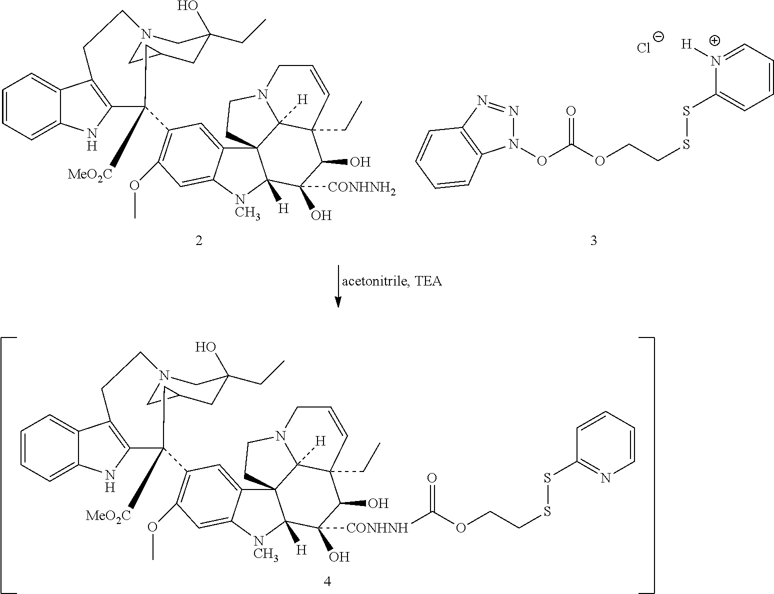

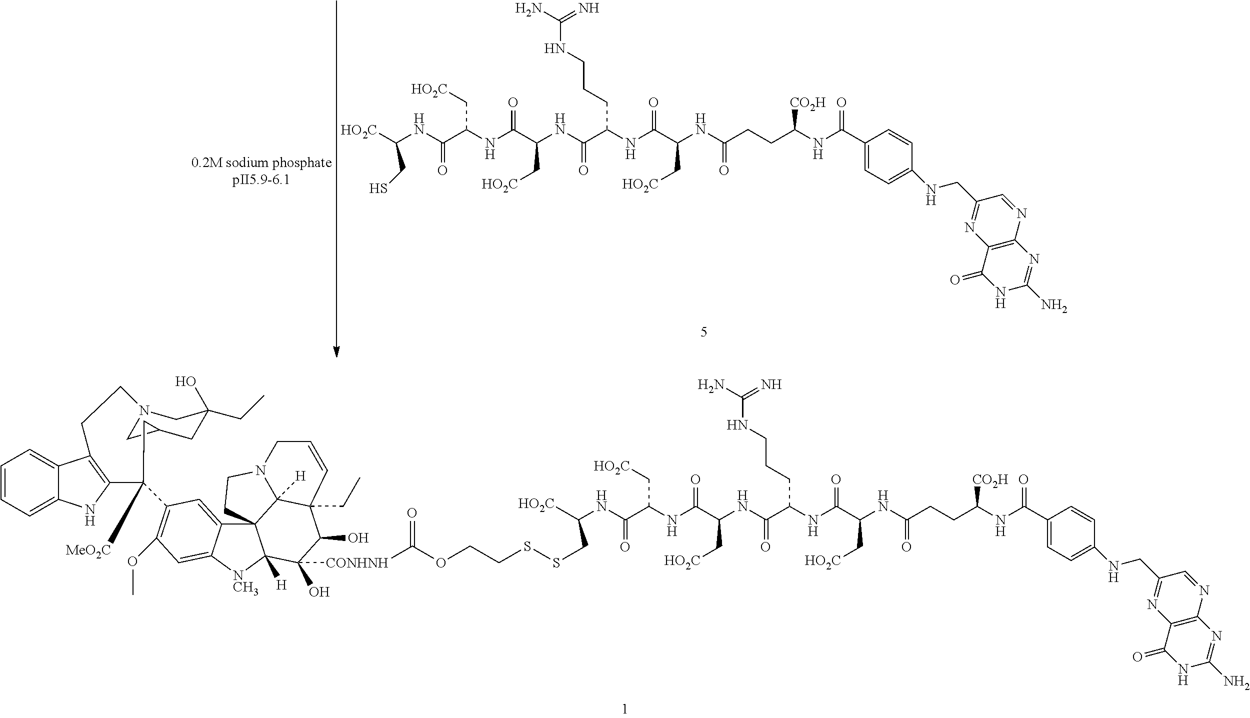

### Step 2 and Step 3 Processes

### Materials

Desacetylvinblastine hydrazide: FW=768.9 g/mol; 20.5 g, 26.7 mmol; Mixed Carbonate (3): FW=384.9 g/mol; 10.7 g, 27.8 mmol; Acetonitrile: q.s.: Triethylamine: FW=101.2 g/mol; 2.67 g, 26.4 mmol; Na₂PO₄.7H₂O: 47.84 g; EC119: 29.9 g 28.6 mmole; 0.5 N HCl: q.s.; WFI: q.s.

### Procedure

Note that all of the water used in this process is water for injection (WFI).

Purge an appropriate vessel with argon. Charge 20.5±0.3 g of des-acetylvinblastine hydrazide; this charge is potency adjusted, i.e., if the potency were 90.0%, the charge would be 22.8 g. Charge 10.7±0.2 g of Mixed Carbonate (potency adjusted). Charge 800±30 mL of acetonitrile and 2.67±0.11 g of triethylamine. Mix under argon at 10-14° C. for 20-28 hours. Take a sample for HPLC (EC145-CMC-AM-0001, version 2.3). The expected result is the ratio of CDSI to hydrazide ≧25:1. If not, continue mixing under argon at 10-14° C. for 2-4 hours and sample again.

Sparge 780-820 mL of water with argon until the dissolved oxygen level is less than 0.9 ppm; record dissolved oxygen level. Dissolve 47.8±0.5 g of sodium phosphate dibasic heptahydrate in the deoxygenated water. To a suitable container, add 29.8±0.5 g of EC119; (charge is potency adjusted). Add the sodium phosphate solution to the EC119 and mix under argon. Measure the solution's pH and adjust the pH to 5.8-6.2 with 0.5 N HCl if necessary.

Add the buffered EC119 solution to the reaction mixture. Mix under argon at 20-25° C. for 60-75 minutes. Take a sample for HPLC (EC145-CMC-AM-0001, version 2.3). If the ratio of EC145 to CDSI ≧25:1, proceed. If not, continue mixing under argon at 20-25° C. and sample again. If the ratio of EC145 to CDSI ≧25:1, proceed. If not, add an additional 1 g of EC119 and mix under argon at 20-25° C. for 30 minutes and sample again.

Prepare 6.9 L-7.1 L of 25 mM phosphate buffer, 185-195 mM NaCl, pH 7.2-7.5 made from water sparged with argon until the dissolved oxygen level is less than 0.9 ppm. Dilute the reaction mixture with this buffer. If the mixture develops more than a faint haze, the product solution needs to be filtered (Whatman Polycap TC75 or TC150, 0.45 or 1.0 micron); this filtration may be done while loading the product onto the Biotage column.

### Liquid Chromatographic Purification

Use a Biotage 150M, C18 cartridge. This size cartridge can accommodate a reaction mixture twice the size of the one currently described.

Column Preparation:

a. Flush the column with
i. 12-13 L of acetonitrile
ii. 12-13 L of 80% acetonitrile and 20% water (v/v)
iii. 12-13 L of 50% acetonitrile and 50% water (v/v)
iv. 12-13 L of 10% acetonitrile and 90% water (v/v)

Purification:

Prepare a 25 mM phosphate buffer, (185-195 mmol) NaCl, pH 7.3-7.5
Sparge the buffer with argon until the dissolved oxygen content is ≦0.9 ppm.
Prepare: 41 L of 10% acetonitrile in buffered saline (v/v); 13 L of 16% acetonitrile in buffered saline (v/v), 52 L of 27% acetonitrile in buffered saline (v/v).
Check the dissolved oxygen content of the mobile phase solutions. If the dissolved oxygen content is greater than 0.9 ppm, sparge the mobile phase with argon or nitrogen until the dissolved oxygen level is ≦0.9 ppm.
Flush the column with 26-27 L of the 10% acetonitrile mobile phase.
Load the product solution onto the column
Elute the product using the following sequence of mobile phases:
i. 13-14 L of the 10% acetonitrile mobile phase.
ii. 13 L of the 16% acetonitrile mobile phase.
iii. 51-52 L of the 27% acetonitrile mobile phase.

Notes: An inline uv detector is helpful; Product should come out starting at 15-19 L of the 27% acetonitrile mobile phase with a bandwidth of 8-13 L.

### Fraction Evaluation

i. HPLC Method EC145-CMC-IP-0001
ii. Passing fraction=≧97.0% EC145 and no impurity ≧0.8%

Post-Run Column Treatment:
The column can be reused once. If the column will be used for a second run, perform ii-iv.
i. Flush column with 12-13 L of 1:1 acetonitrile-water.
ii. Flush column with 20-22 L of acetonitrile
iii. Repeat column preparation steps ii-iv

### Ultra-Filtration

Sparge q.s. water with argon or nitrogen until the dissolved oxygen level is less than 0.9 ppm. Passing chromatography fractions are combined and diluted with an equivalent volume of sparged water. Assemble an ultra-filtration apparatus using a Millipore regenerated cellulose membrane with nominal MW cutoff of 1000 (cat#CDUF002LA) and rinse it with 9 L of deoxygenated water. Start ultra-filtration of the product solution. Maintain a backpressure of 30-50 psi. Continue ultra-filtration until the retentate volume is 2 to 3 L. Add 11 to 12 L of deoxygenated water. Continue ultra-filtration until the retentate volume is 2 to 3 L. Add 11 to 12 L of deoxygenated water. Continue ultra-filtration until the retentate volume is 2 to 3 L. Add 8 to 10 L of deoxygenated water. Continue the ultra-filtration until the retentate volume is 2 L. The ultra-filtration endpoint must be determined by analyzing a sample of the retentate via GC and concentration. The specification is ≦50 micrograms of acetonitrile per milligram of EC145. If not achieved, perform another cycle of the ultra-filtration.

The API solution's concentration must be adjusted so that the packaged material is 6 to 12 mg/mL. At the completion of the ultra-filtration, the apparatus will be rinsed with 1 liter of water. Therefore, continue ultra-filtration or add water as necessary. Once the product solution is out of the ultra-filtration apparatus, rinse the ultra-filtration apparatus with 1 L of deoxygenated water and combine with the product solution.

After the rinse is combined with the product solution, this solution must be filtered through a 0.2 micron absolute filter, and this filtrate is packaged (performed under an inert atmosphere).

A typical yield of isolated product is 50-60% of the theoretical maximum.

### Example 3

### Steps 2 and 3 of the EC145 Process

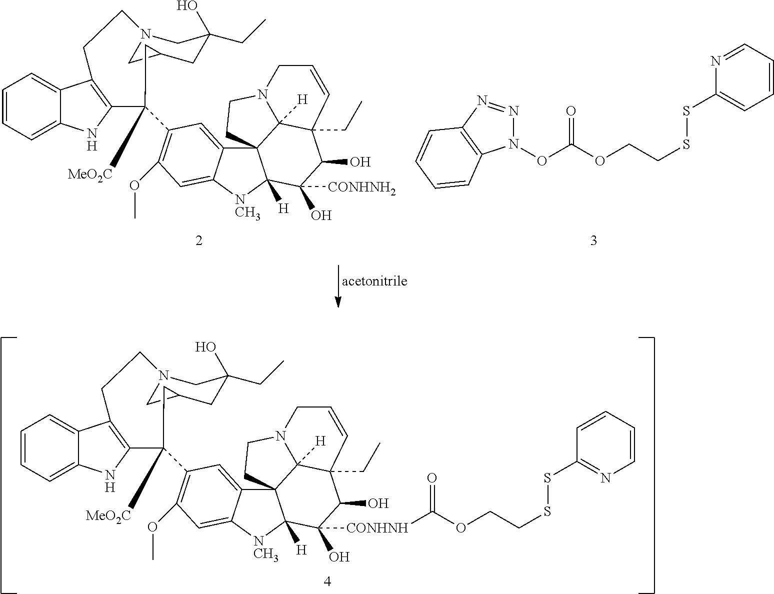

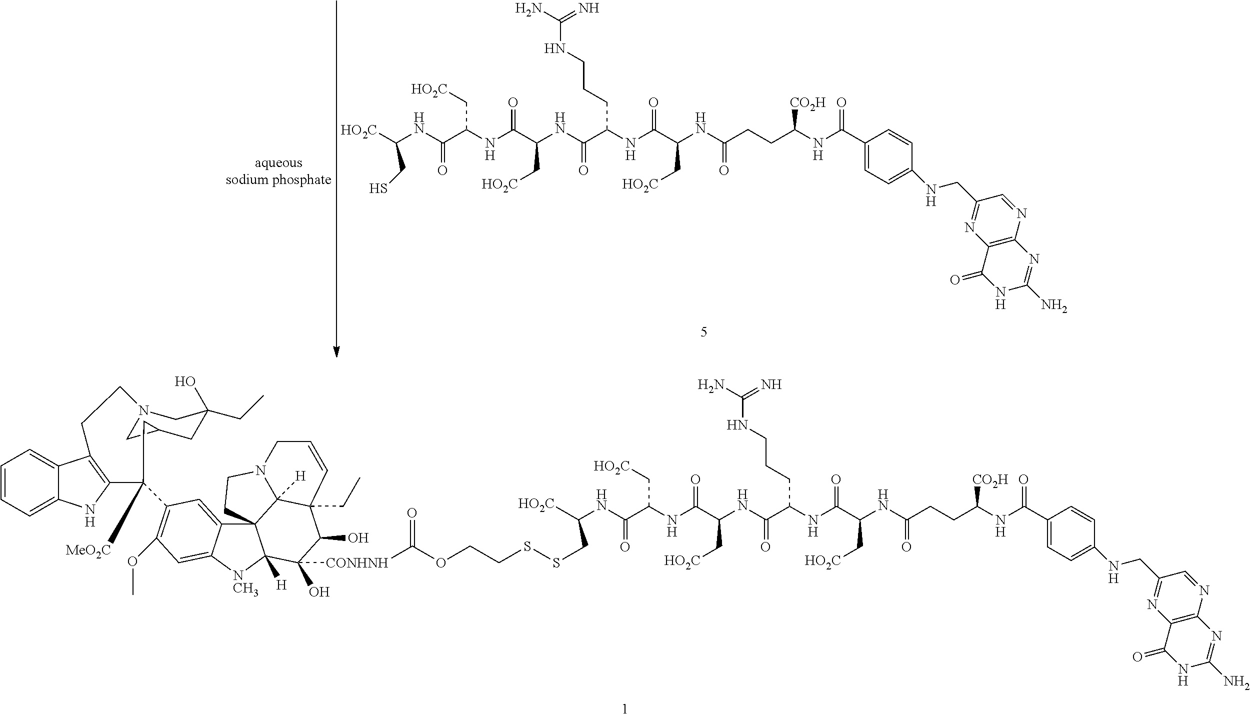

### Step 2 and Step 3 Processes

### Materials

Desacetylvinblastine hydrazide: FW=768.9 g/mol; 1.00 g, 1.30 mmol; Mixed Carbonate (3): FW=348.4 g/mol; 0.445 g, 1.28 mmol; Acetonitrile: q.s.: Na₂PO₄: 1.10 g; EC119: 1.46 g 1.40 mmole; 0.5 N HCl: q.s.; WFI: q.s.

### Procedure

Note that all of the water used in this process is WFI.

Purge an appropriate vessel with argon. Charge 1.00±0.02 g of desacetylvinblastine hydrazide; this charge is potency adjusted, i.e., if the potency were 90.0%, the charge would be 1.11 g. Charge 0.445±0.005 g of Mixed Carbonate (potency adjusted). Charge 46±1 mL of acetonitrile. Mix under argon at 10-20° C. for 22-23 hours. Take a sample for HPLC (EC145-CMC-AM-0001, version 2.3). The expected result is the ratio of CDSI to hydrazide ≧20:1. If not, continue mixing under argon at 10-20° C. for 2-3 hours and sample again.

Sparge 41 mL of water with argon until the dissolved oxygen level is less than 0.9 ppm; record dissolved oxygen level. Dissolve 1.10±0.0.07 g of dibasic sodium phosphate in the deoxygenated water. To a suitable container, add 1.46±0.03 g of EC119; (charge is potency adjusted). Add the sodium phosphate solution to the EC119 and mix under argon. Measure the solution's pH and adjust the pH to 5.9-6.3 with 0.5 N HCl if necessary.

Add the buffered EC119 solution to the reaction mixture. Mix under argon at 20-25° C. for 60-75 minutes. Take a sample for HPLC (EC145-CMC-AM-0001, version 2.3). If the ratio of EC145 to CDSI ≧20:1, proceed. If not, continue mixing under argon at 13-23° C. and sample again. If the ratio of EC145 to CDSI ≧20:1, proceed. If not, add an additional 1 g of EC119 and mix under argon at 13-23° C. for 30 minutes and sample again.

Prepare 399-401 mL of 0.02 mM citrate buffered, aqueous 0.1 M NaCl, pH 5.7-6.5 made from water sparged with argon until the dissolved oxygen level is less than 0.9 ppm. Dilute the reaction mixture with this buffer. If the mixture develops more than a faint haze, the product solution needs to be filtered (Whatman Polycap TC75 or TC150, 0.45 or 1.0 micron); this filtration may be done while loading the product onto the Biotage column.

### Liquid Chromatographic Purification

Use a Biotage cartridge packed with polystyrene-divinylbenzene polymeric resin.

Column Preparation:

b. Flush the column with
i. acetonitrile
ii. 50% acetonitrile and 50% water (v/v)
iii. 10% acetonitrile and 90% water (v/v)

### Purification:

Prepare a citrate buffered, aqueous sodium chloride solution, pH 6.0-6.6.
Sparge the buffer with argon until the dissolved oxygen content is ≦0.9 ppm.
Prepare: 12% acetonitrile in the buffered, aqueous sodium chloride (v/v); 16% acetonitrile in buffered, aqueous sodium chloride (v/v), 22.5% acetonitrile in buffered, aqueous sodium chloride (v/v).
Check the dissolved oxygen content of the mobile phase solutions. If the dissolved oxygen content is greater than 0.9 ppm, sparge the mobile phase with argon or nitrogen until the dissolved oxygen level is ≦0.9 ppm.
Flush the column with the 10% acetonitrile mobile phase.
Load the product solution onto the column
Elute the product using the following sequence of mobile phases:
iv. the 16% acetonitrile mobile phase.
v. the 22.5% acetonitrile mobile phase.

### Fraction Evaluation

iii. HPLC Method EC145-CMC-IP-0001
iv. Passing fraction=≧97.0% EC145 and no impurity ≧0.8%

### Ultra-Filtration

Sparge q.s. water with argon or nitrogen until the dissolved oxygen level is less than 0.7 ppm. Passing chromatography fractions are combined and diluted with an equivalent volume of sparged water. Assemble an ultra-filtration apparatus using a Millipore regenerated cellulose membrane with nominal MW cutoff of 1000 and rinse it with deoxygenated water. Start ultra-filtration of the product solution. Maintain a backpressure of 30-50 psi. Continue ultra-filtration until the retentate volume is approximately 20% of the original volume. Add deoxygenated water to the retentate. Continue ultra-filtration until the retentate volume is approximately 20% of the original volume. Add deoxygenated water to the retentate. Continue ultra-filtration until the retentate volume is approximately 20% of the original volume. Add deoxygenated water to the retentate. Continue the ultra-filtration until the retentate volume is approximately 20% of the original volume. The ultra-filtration endpoint must be determined by analyzing a sample of the retentate via GC and concentration. The specification is ≦50 micrograms of acetonitrile per milligram of EC145. If not achieved, perform another cycle of the ultra-filtration.

The API solution's concentration must be adjusted so that the packaged material is about 12 mg/mL. At the completion of the ultra-filtration, the apparatus will be rinsed with water. Therefore, continue ultra-filtration or add water as necessary. Once the product solution is out of the ultra-filtration apparatus, rinse the ultra-filtration apparatus with deoxygenated water and combine with the product solution.

After the rinse is combined with the product solution, this solution must be filtered through a 0.2 micron absolute filter, and this filtrate is packaged (performed under an inert atmosphere).

A typical yield of isolated product is 50-60% of the theoretical maximum.

What is claimed is:

1. A process for preparing EC145 comprising the step of treating a compound of formula 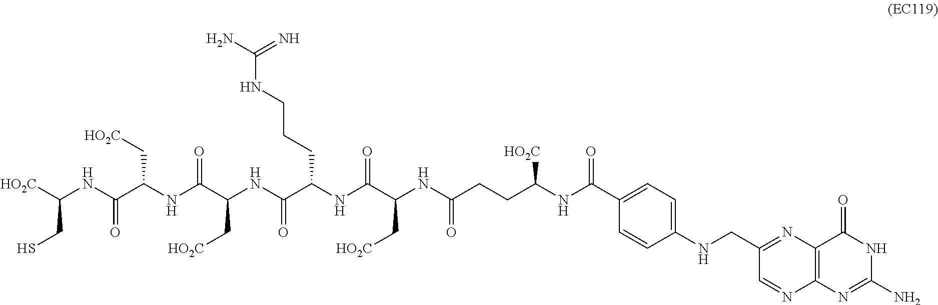 [SMILES: N=C(N)NCCC[C@H](NC(=O)[C@H](CC(=O)O)NC(=O)CC[C@H](NC(=O)c1ccc(NCc2cnc3nc(N)[nH]c(=O)c3n2)cc1)C(=O)O)C(=O)N[C@@H](CC(=O)O)C(=O)N[C@@H](CC(=O)O)C(=O)N[C@@H](CS)C(=O)O]
with a compound of formula, 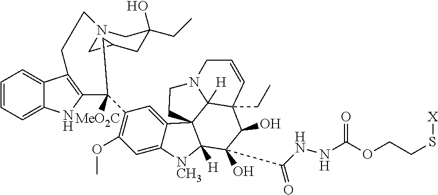
wherein X is alkylsulfonyl in the presence of an aqueous buffer of pH less than 8.
